(19) United States
(12) Patent Application Publication
(10) Pub. No.: US 2022/0298322 A1
(43) Pub. Date: Sep. 22, 2022

(54) Method of making a porous sponge-like formulation, a porous sponge-like formulation, use of porous sponge-like formulation and a product comprising the foamed sponge-like formulation

(71) Applicant: ETH Zürich, Zürich (CH)

(72) Inventors: Erich Windhab, Hemishofen (CH); Loredana Malafronte, Hisings Backa/Gothenburg (SE); Socrates Foschini, Zürich (CH); Judith Wemmer, Zürich (CH)

(21) Appl. No.: 17/638,566

(22) PCT Filed: Aug. 30, 2019
(86) PCT No.: PCT/EP2019/000250

§ 371 Date: Feb. 25, 2022

(51) Int. Cl. C08J 9/30; A61L 15/32; A61L 15/42; C09K 3/32; B01J 20/24; B01J 20/28

(52) U.S. Cl. CPC C08J 9/30; A61L 15/32; A61L 15/425; C09K 3/32; B01J 20/24; B01J 20/28045; B01J 20/28069; C08J 2201/05; C08J 2205/044; C08J 2205/05; C08J 2207/10; C08J 2207/12; C08J 2389/00; B01J 2220/4856

(57) ABSTRACT

The present invention relates to a method of making a porous sponge-like formulation that can well absorb water, oil and organic solvents separately or combined. Methods of preparing said formulation and its use in medical, pharmaceutical, biotechnological, chemical as well as in wound care, home care, (agro-)environmental and construction material applications are also provided.

### STATE OF THE ART

Artificial sponge-like structures are being applied for various technical challenges in food, cosmetics, or pharma industries as well as for energy storage, waste water treatment or in home care, wound care as well as agricultural and construction material applications. The high porosity of these sponge structures results in a high surface-to-volume ratio and thus in the possibility to interact with the surrounding, in a low density and thus light weight and in the ability to passively or actively take up liquids. The replacement of synthetic polymers by biopolymers as bulk material enhances the biodegradability, renewability and recyclability of related products/materials.

Various processes are described to generate biodegradable sponge-like structures for absorption based separation applications, e.g. for oil-water separation.

Both (A) CN108273476 A (alginate-protein sponge) as well as (2) WO2015056273 A1 (seaweed-polysaccharide sponge) or (3) Duan, Bo, et al. “Hydrophobic modification on surface of chitin sponges for highly effective separation of oil” ACS applied materials & interfaces 6.22 (2014): 19933-19942 (chitin sponge) prepare mixtures with biopolymers, perform cross-linking with chemical additives, followed by freeze-drying/lyophilization to generate a dry porous oil-absorbing structure.

Aerogels based on cellulose fibers can be amphiphile absorbents, hence absorbing water, oil or organic solvents, as e.g., described by (4) Jiang, Feng, and You-Lo Hsieh. “Amphiphilic superabsorbent cellulose nanofibril aerogels”, Journal of Materials Chemistry A 2.18 (2014): 6337-6342. Also these porous structures were produced by freeze-drying.

Other processes for the production of absorbents include chemical polymerization, washing with solvents followed by vacuum drying, e.g., (5) Zhu, Haiguang, et al. “A robust absorbent material based on light-responsive superhydrophobic melamine sponge for oil recovery.” Advanced Materials Interfaces 3.5 (2016): 1500683. (6) M. Betz, C. A. Garcia-Gonzalez, R. P. Subrahmanyam, I. Smirnova, U. Kulozik, Preparation of novel whey protein-based aerogels as drug carriers for life science applications, The Journal of Supercritical Fluids, Volume 72, 2012, Pages 111-119, ISSN 0896-8446 and (7) Selmer, Ilka, et al. “Development of egg white protein aerogels as new matrix material for microencapsulation in food.” The Journal of Supercritical Fluids 106 (2015): 42-49 describe the production of pure protein aerogels stable in water and with the ability to absorb water. These protein aerogels are produced by applying a heating step followed by freeze-drying or supercritical carbon dioxide drying.

(8) V. Perez-Puyana, M. Felix, A. Romero, A. Guerrero, Development of eco-friendly biodegradable superabsorbent materials obtained by injection moulding, Journal of Cleaner Production, Volume 198, 2018, Pages 312-319, ISSN 0959-6526 try to apply a more scalable injection moulding process to produce absorbents based on proteins. The process however requires the addition of nanoclay particles and plasticizer.

All production processes for absorbents based on proteins described in literature require either time-, energy and cost-intensive drying methods such as freeze-drying, supercritical carbon dioxide drying or vacuum drying and/or the utilization of additives such as chemical cross-linking agents, fillers, etc. Further, the so far described sponge-like products received from such described production processes preferably absorb either water or oil based liquids.

### SUMMARY OF THE INVENTION

The invention relates in general to a method of making a porous sponge-like formulation that can absorb water, oil and organic solvents, said method comprising the steps:
Preparing a protein dispersion in water;
Dispersing gas in the protein dispersion to form a foam structure;
Optionally moulding into a shape;
Expansion of the foam structure;
Volumetric electromagnetic heating;
Optionally drying; and
Optionally cutting into pieces.

The invention further relates to a porous sponge-like formulation comprising protein, preferably obtained by a method as described herein.

The invention further relates to the use of a porous sponge-like formulation as described herein in a non-food product.

The present invention relates to a method of making a porous sponge-like formulation that can absorb water and oil, said method comprising the steps:
Preparing a 5-60 wt % protein dispersion in aqueous liquid, preferably in water;
Dispersing gas in the protein dispersion to form a foam structure;
Optionally moulding or shaping to form a foam structure;
Expansion of the foam structure;
Volumetric heating induced water evaporation and protein denaturation;
Optionally drying; and
Optionally cutting into pieces.

In some embodiments, 10-50 wt % protein dispersion is prepared in water, preferably 15-45 wt % protein dispersion.

In some embodiments, the protein is a globular protein, preferably a plant protein.

In some embodiments, the protein dispersion is a homogenous dispersion.

In some embodiments, the protein dispersion is a whey protein isolate dispersion.

In some embodiments, the protein dispersion further comprises fibre, for example, fibrillated or crystalline cellulose.

In some embodiments, the protein dispersion further comprises plasticisers for example sugar, and/or hydrocolloid.

In some embodiments, the protein dispersion further comprises fillers, for example clay particles.

In some embodiments, gas is dispersed in the protein dispersion using a rotating membrane foaming device.

In some embodiments, said foam structure has a gas volume fraction of 10-90 vol %, preferably 40-80 vol %, most preferably 60-75 vol %.

In some embodiments, the foam structure is increased to above the protein denaturation temperature.

In some embodiments, the temperature gradient between the core and the surface layer of the foam structure is between −0.1 and 0.3, preferably between −0.1 and 0.2, more preferably between −0.1 and 0.1.

In some embodiments, heating is volumetric preferably through application of electromagnetic waves, preferably microwave power.

In some embodiments, a vacuum is applied before and/or during drying which is between 10-800 mbar, preferably 50-500 mbar, more preferably between 100-300 mbar.

In some embodiments, said porous sponge-like formulation has open pores having an average pore diameter of up to 500 microns, preferably up to 200 microns.

The invention further relates to a porous sponge-like formulation that can absorb water and oil and comprises protein, obtained by a method as described herein.

The invention further relates to a foamed porous sponge-like formulation that can absorb water, oil and organic solvents and comprises protein, wherein the porous formulation has a water content <15 wt % after drying and has a porosity of between 10-95 vol %, preferably 80-95 vol %; and wherein said formulation can absorb water and oil without disintegrating or dissolving to an extent of less than 10 wt %.

In some embodiments, the moisture content of the porous sponge-like formulation is less than 60 wt %, preferably less than 20 wt %, more preferably less than 10 wt %.

In some embodiments, said formulation further comprises fibres and/or other biopolymers, for example polysaccharides.

In some embodiments, said formulation is capable of absorbing water, oil and organic solvent at substantially the same velocity.

In some embodiments, said formulation is capable of absorbing water at a velocity of up to 2.2 mm/s, preferably up to 5 mm/s at 0-100° C. and without structure disintegration

In some embodiments, said formulation is capable of absorbing water with a temperature of 0-100° C. to an extent that up to 140% of pore volume is filled with water due to additional structural swelling effects.

In some embodiments, said formulation is capable of absorbing oil at a velocity of up to 1.5 mm/s, preferably up to 5 mm/s at 0-200° C. without structure disintegration or filling the formulation structure up to 90% of pores, preferably up to 95% of pores, most preferably up to 100% of the pore volume with oil with a temperature of 0-200° C.

In some embodiments, said formulation is elastic-plastically deformable after absorption of water and is elastic-brittle in substantially dry state and after absorption of oil or ethanol, methanol, acetone, dimethyl sulfoxide and toluene.

The invention further relates to use of the porous sponge-like formulation as described herein in a product, for example a wound care related secretion absorber material.

The invention further relates to use of the porous sponge-like formulation as described herein in a product, for example a as filter and/or absorber for the cleaning of watery and/or oil-based fluid systems and/or water/oil mixtures.

The invention further relates to use of the porous sponge-like formulation as described herein in a product, for example for medical surgery applications to enable larger amounts of blood or secretion fluid uptake.

The invention further relates to use of the porous sponge-like formulation as described herein in a product, for example for cosmetics applications with water/oil-based liquid and skin care ingredients or skin cleaning fluids release from and/or uptake into the sponge-like product.

The invention further relates to use of the porous sponge-like formulation as described herein in a product, for example for the encapsulation of active/functional ingredients from the categories: flavors, aromas, micronutrients, antioxidants, agro-chemicals, chemicals, washing agents, drugs, pre-/probiotic cultures, skin care components, cleaning components.

The invention further relates to use of the porous sponge-like formulation as described herein in a product, for example as template for cell culturing.

The invention further relates to use of the porous sponge-like formulation as described herein in a product, for example applied in one of the areas of medicine, pharmacology, biotechnology, chemistry as well as in home care, wound care, (agro-) environmental and construction material applications.

The invention further relates to use of the porous sponge-like formulation as described herein in a product, for example as immobilization carrier for microorganisms in biotechnological, pharmaceutical and/or medical applications.

The invention further relates to a wound care related product comprising the porous sponge-like formulation as described herein.

### DETAILED DESCRIPTION OF THE INVENTION

### Definitions

The following definitions are provided for the technical features used throughout the specification.

Sponge-like denotes a porous structure with 5-95% porosity and up to 100% of open pore or pore channel structure, which allows for the passive or active absorption of a liquid into the porous structure.

Heat induced expansion denotes an increase of aerated product pore volume by more than 25%, preferably more than 50% upon heating and vapor pressure generation.

Protein denaturation through heating denotes unfolding or dissociation of the protein structure induced by heat, followed by re-association and/or aggregation. The transition from native to denatured state is associated with an alteration in secondary and tertiary structure of the protein through rupture of hydrogen bonds, ionic interactions and cleavage of disulfide bridges.

Volumetric heating denotes heating of an entire volume (center to surface) of a structure or product, e.g., by application of electromagnetic waves, such as microwaves, which penetrate into the structure resulting in heat dissipation. This is in contrast to heating by convection or conduction, which leads to heating of the surface and subsequent heat transfer from the surface toward the center.

Electromagnetic waves or radiation denote waves of an electromagnetic field propagating through space and carrying electromagnetic radiant energy. It includes radio waves, microwaves, infrared, visible light, ultraviolet, X-rays and gamma-rays.

The temperature gradient or relative temperature gradient denotes the temperature difference between the geometric center of the cross-section (in radial direction) and the temperature in the surface layer divided by the center temperature (Temp. gradient=(Tcenter−Tsurface)/Tcenter. It was assessed by measuring the temperature in the geometric center and in the surface layer at maximum half the radius of the moulded or shaped foam structure by means of fiber-optic temperature sensors.

Disintegrating means breaking into more than one piece, for example after the porous sponge-like formulation is immersed in a liquid.

Protein denotes plant and/or animal based bio-macromolecules, consisting of one or more long chains of amino acid residues. A protein is typically a polymer consisting of 50 or more amino acid residues linked by peptide bonds. Examples of proteins of the invention are whey protein, egg white protein, pea protein, and soy protein.

Fibre denotes non-starch polysaccharides with 10 or more monomeric units. The solubility of a fibre is determined by the relative stability of the ordered and disordered form of the polysaccharide. Molecules that fit together in a crystalline array are likely to be energetically more stable in solid state than in solution. Hence, linear polysaccharides, i.e., cellulose, tend to be insoluble (non-soluble), while branched polysaccharides or polysaccharides with side chains, such as pectin or modified cellulose, are more soluble. Hence, non-soluble fibre denotes fibre with low or no solubility in water. This might however contain residues of soluble fibre due to the production/extraction process. Soluble fibre denotes dietary fibre with high solubility such as pectin. Examples of non-soluble fibres of the invention are cellulose fibre, for example citrus fibre, microfibrillated cellulose or microcrystalline cellulose.

When a composition is described herein in terms of wt %, this means a mixture of the ingredients on a dry basis, unless indicated otherwise.

Porosity denotes the fraction of pore volume in the entire volume of the porous sponge-like formulation, wherein the pore volume denotes the accumulate volume of all pores.

Brittle denotes fracturing upon exceeding the elastic deformation limit without undergoing plastic deformation.

Elastically and plastically deforming or elastic-plastic deformation of porous solids denotes elastic deformation followed by plastic yielding of the structure and stands in contrast to brittle crushing of the structure. The elastic part of the deformation is typically reversible, while the plastic part is typically irreversible.

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

As used herein the term “about” means approximately, in the region of, roughly, or around. When the term “about” is used in conjunction with a numerical value or range, it modifies that value or range by extending the boundaries above and below the numerical value(s) set forth. In general, the term “about” is used herein to modify (a) numerical value(s) above and below the stated value(s) by 10%.

Substantially dry denotes drying to an extent that the water content is below 12 wt %.

Substantially the same velocity during absorption of oil and water denotes a relative difference in velocity of not more than 200% at same liquid viscosity, given that water and oil might not show the same wettability towards the porous sponge-like formulation nor the same surface tension toward air.

Method of Making a Porous Sponge-Like Formulation

The formulation is made by foaming a highly-concentrated protein dispersion followed by volumetric heating and drying. Depending on the viscosity of the protein dispersion, the foaming step may be performed by extrusion foaming, membrane foaming or other foaming techniques. The foam structure is optionally moulded or shaped followed by heating and optionally drying by controlled volumetric heating through superposition of electromagnetic heating, e.g., microwave, and hot air. Volumetric heating such as generated by microwave results in quick steam generation and accumulation in the foam bubbles causing expansion of the foam structure. At the same time, heating causes fast denaturation of the proteins at the bubble interface and in the foam lamellae. Controlled microwave power input and hot air temperature allow for a generation of a homogeneous temperature distribution throughout the structure. This leads to a homogeneous expansion, denaturation and continuous transport of moisture from the material to the surface and surrounding and thus to a late crust formation. The foam bubbles expand to an extent that they coalesce and form open channels throughout the structure. The resulting dry open-porous and stiff structure adsorbs water and/or oil to comparable extents given by the accessibility of both hydrophilic and hydrophobic parts of the intrinsically amphiphilic proteins. Other heating methods can be used, which allow for volumetric heating, such as infrared heating and Ohmic heating.

Porous Sponge-Like Formulation

The dry foam sponge material of the invention, upon contact with a liquid phase (aqueous or oil phase), can take up the liquid and release it again upon applying a stress or upon suction to it. The dried foam, denoted also as dry sponge, is preferably made out of globular proteins that denature upon heat treatment, such as whey protein or whey protein isolate. Other globular proteins can also be used. The dry sponge adsorbs the liquid without disintegrating the sponge structure and may thus be applied as a dried foamed material that is able to take up and release a liquid and hold it and release it upon mechanical stress impact.

The sponge material can absorb both aqueous and oily phases. The sponge mechanics can be modulated by tailoring the density or by adding additional fibres and/or other additives.

Product

The material can be applied in various products to introduce some liquid sucking, holding and controlled release characteristics. The latter can relate to the fluid and/or functional components added or contained in the fluid.

This includes the four main categories of:

(1) Using the product for sucking and/or immobilizing a liquid or liquid mixture:
This can be relevant for cleaning or separation processes including the cleaning of wound surfaces from secreted wound fluid or blood. This further includes applications where the product, also in refined sponge-particle form can be added to a matrix material (e.g. construction material) from which contained liquid shall be taken up for stiffening the matrix material.
(2) Release of product entrapped liquid or of components contained in such liquid:
This can be of interest for the release of functional components to trigger a physiological, biological or chemical reaction in the surrounding/surrounding material (e.g. wound healing support; disinfection/sanitizing; cosmetics function; cleaning function; perfume or skin cream delivery
(3) Use of product structure as template for culturing:
This is expected to be relevant for cell culturing (skin, organs, meat/meat replacers)
(4) Use for immobilizing microorganisms or cells to enable improved biotechnological processing (e.g. for pharmaceutical or medical applications).

### EXAMPLES

### Example 1

Whey protein sponge production and comparison to pure hot air drying About 40 wt % whey protein isolate was dispersed in tap water and hydrated overnight. The dispersion was foamed by dispersing gaseous nitrogen in the protein dispersion with a rotating membrane foaming device. The resulting protein foam had a gas volume fraction of 70 vol % and a number weighted mean bubble size d50, 0=54 um with a span of 1.28 (measure for bubble size distribution width, defined as (x90, 0−x10, 0)/x50, 0).

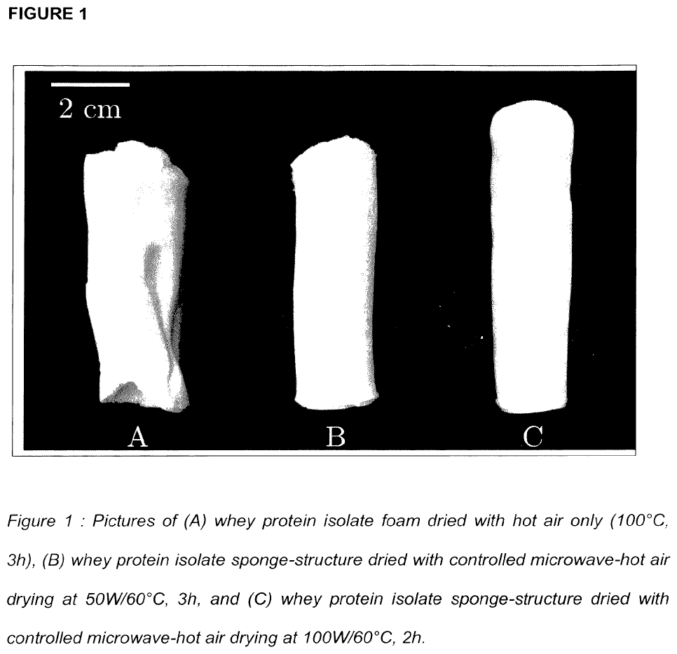

About 24 mL of the foam was filled into cylindrical transparent polypropylene moulds with a diameter of 27.5 mm and a height of 86 mm. The samples (4 samples per trial) were dried at a microwave power of 100 W and a hot air temperature of 60° C. for over 2 hours or at a microwave power of 50 W and a hot air temperature of 60° C. over 3 hours. The resulting dry foam with a diameter of 20 mm and a height of 70-85 mm, shown in FIGS. 1 (B) and (C), can be removed from the mould and further processed, for example by cutting into pieces.

For comparison, FIG. 1 (A) shows the same foam dried without superposition of microwave only with hot air at a temperature of 100° C. over 3 hours. It has a heterogeneous, wrinkled, partly shrunken structure with a darker outer crust.

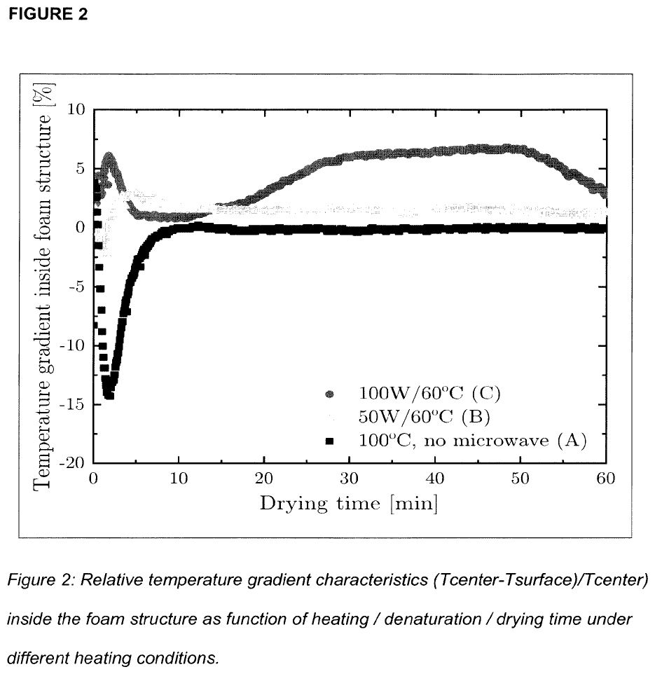

Measuring the temperature of the foam structure during heating and drying in the radial center and in the surface layer, showed the importance of a homogeneous temperature gradient during the heating and drying process. FIG. 2 shows the time-dependency of the relative temperature gradient inside the foam structure (in %), defined as temperature difference between the geometric radial center and the sample surface layer related to the center temperature. Pure hot air drying leads to a highly negative temperature gradient, meaning that the surface heats up much faster than the center. In contrast, superposition of microwave caused a faster heating of the core and in particular a significantly more even heating throughout the entire cross section of the foam product. This results accordingly in an even expansion, protein denaturation and water transport during drying and thus minimises evaporation-induced uneven shrinkage resulting in the generation of a homogeneous porous structure.

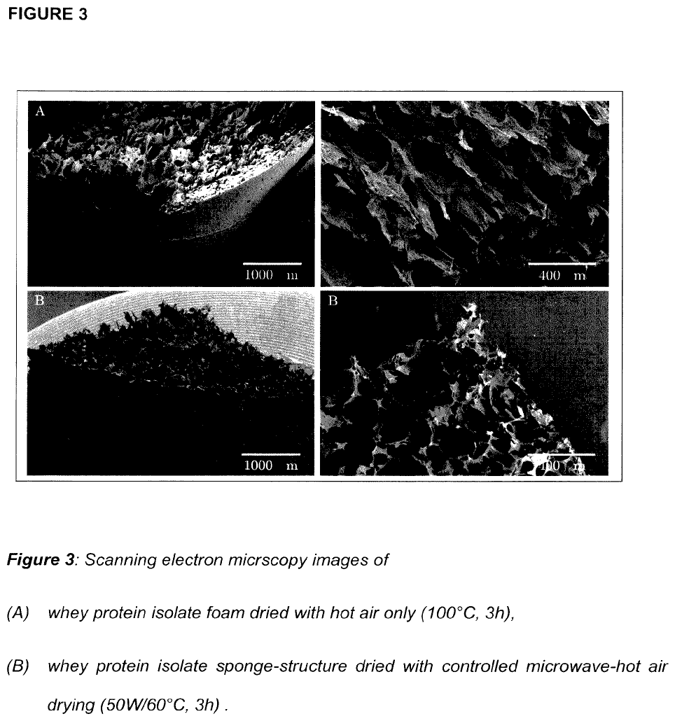

Scanning electron microscopy images of the same samples (A) and (B) shown in FIG. 3 reveal a denser crust and a sheet-like structure when drying with hot air only (A) and an open-porous surface and pore structure with spherical pores when drying with superimposed microwave and hot air (B). The spherical pores are foam bubbles retained throughout the heating and drying process. On top of the structural dependency from the drying process a more even protein denaturation distribution across the sample cross section has to be counted with in case of the coupled Microwave/convection drying.

### Example 2

Whey Protein Porous Sponge-Like Formulation Absorbing Water and Oil

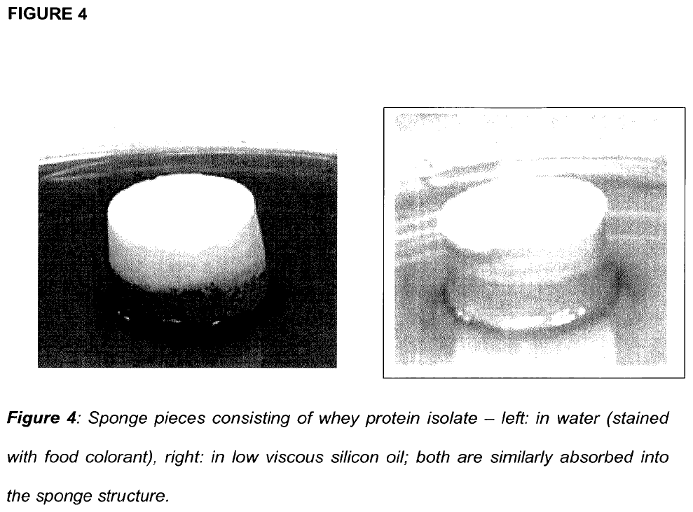

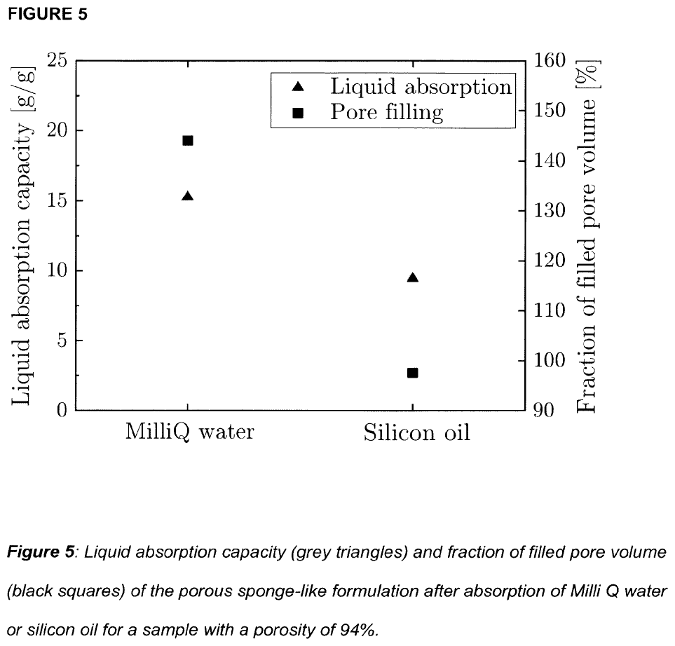

A sponge piece of 20 mm height of sample (C) (100 W/60° C.) in Example 1, with a density of 0.09 g/cm³, shown in FIG. 4 in water (left, stained with food colorant) and in oil (right), absorbed 15 g water ((η=1 mPas; ρ=1.0 g/cm³) and 9 g low-viscous silicon oil (η=3 mPas; ρ=0.9 g/cm³) per g sample at a velocity of approximately 2.2 mm/s for water and approximately 1.5 mm/s for oil. Assuming a solid density of whey protein isolate of 1.4 g/cm³, the density of the dry porous structure of 0.09 g/cm³ corresponds to a porosity of 94%. Hence, at the measured oil absorption capacity, 94% of the pore volume in the dry porous structure must get filled with oil. In contrast, the water filled up over 140% of the pore volume (see FIG. 5), meaning that the porous sponge-like formulation swells upon absorption of water.

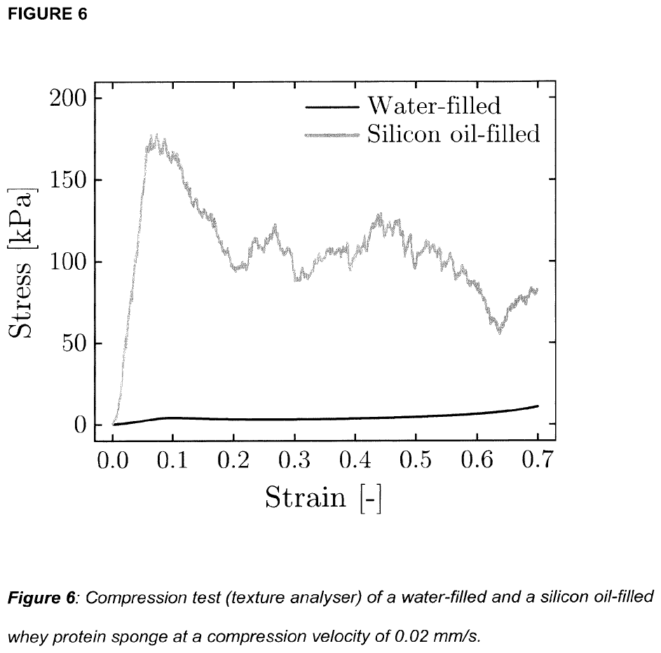

Absorption of water into the whey protein sponge structure causes softening, whereas the sponge remains brittle and stiff upon absorption of oil. FIG. 6 shows the mechanical properties in compression of a water-filled and an oil-filled whey protein sponge at a compression velocity of 0.02 mm/s.

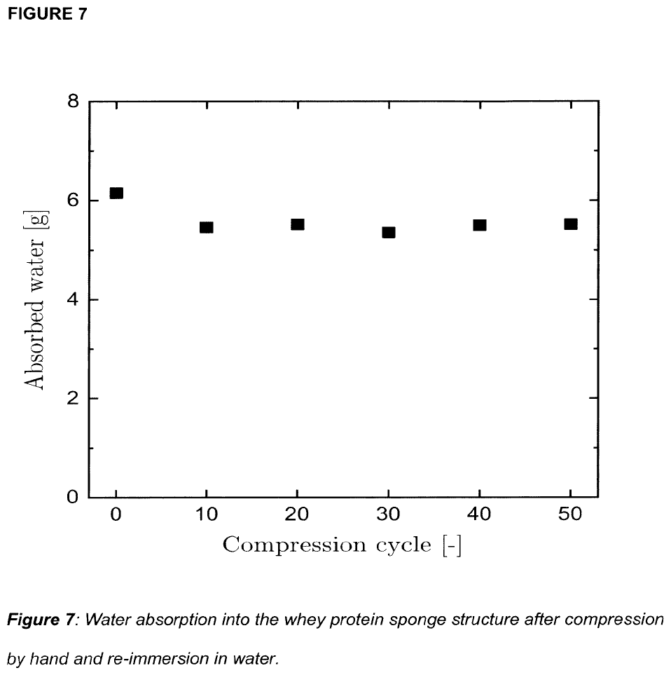

As the sponge softens upon absorption of water, the water can be pressed out, e.g., by hand, and the sponge can be refilled. The weight of absorbed water decreased by not more than 15% over 50 compression and re-absorption cycles, as shown in FIG. 7.

A sponge filled with oil cannot be compressed and re-filled due to the brittle structure. The oil could however be removed by suction, e.g., by vacuum. Alternatively, the sponge can be softened by absorption of water, subsequently the water is squeezed out and the sponge is immersed in oil. Thus, the sponge structure is soft and elastic and the absorbed oil can be squeezed out by hand. This porous sponge-like formulation can be used for example for carrying liquid products (water based cosmetics i.e. body lotions and perfumes), for water holding fertilizer pellets, or as absorbent for cleaning.

### Example 3

Production of Soy Protein Sponge

About 18 wt % soy protein isolate was dispersed in tap water and hydrated overnight; foamed with a kitchen machine (Kitchen Aid) to reach a gas volume fraction of approximately 20 vol %; the foam was distributed onto a Teflon plate in portions of 2 table spoons and dried at 50 W and 60° C. over 1 hours. The resulting stiff sponge structure absorbed water, oil and organic solvents separately, combined or in series without disintegrating and softened when filled with water.

### Example 4

Whey Protein Sponge Reinforced with Citrus Fibres

About 40 wt % whey protein isolate and 5 wt % citrus fibres were dispersed in tap water and hydrated overnight. The dispersion was foamed in a kitchen machine (Kitchen Aid) to reach a gas volume fraction of 40 vol %. Approximately 24 mL of the foam were filled into cylindrical transparent polypropylene moulds with a diameter of 27.5 mm and a height of 86 mm. The samples were dried at a microwave power of 100 W and a hot air temperature of 60° C. over 2 hours. The resulting dry sponge absorbs both water, oil and organic solvent and is stiffer and stronger compared to the pure whey protein sponge.

### Example 5

Whey Protein Sponge Filled with Agar Agar Solution

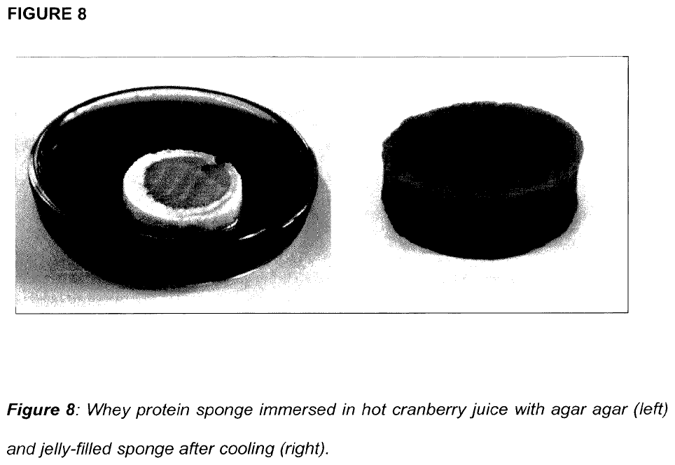

200 mL cranberry juice (for colour contrast) were mixed with 0.7 g agar agar powder, heated to boiling for 1 min. A whey protein sponge produced as described in Example 2 was soaking in the hot cranberry juice-agar agar mixture. The juice was immediately absorbed into the sponge structure (FIG. 8). The filled sponge was cooled for 4 h at 4° C. to cause gelation of the cranberry juice-agar agar mixture.

For comparison, cranberry juice gel was produced with the same concentration of agar agar powder by moulding into a plastic beaker and cooling. The cranberry juice gel was not self-sustaining without the mould.

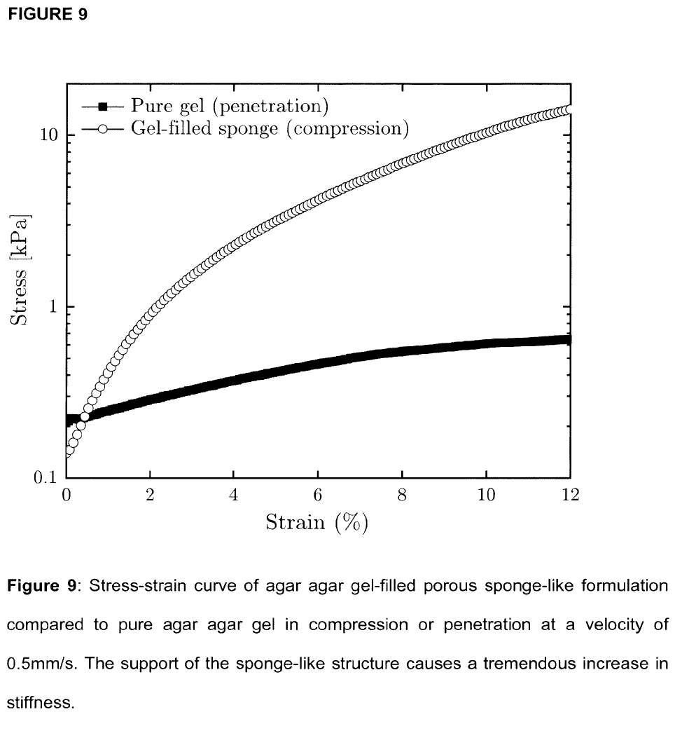

The stiffness of the gel-filled sponge and the pure gel was compared by texture analysis by compression and penetration, respectively, as shown in FIG. 9 at a velocity of 0.5 mm/s. Although the sponge structure makes up below 10 wt % of the gel-filled sponge (>90 wt % cranberry juice gel), the Young's modulus, a measure of stiffness, increases from approximately 35 Pa to 1500 Pa compared to the pure gel. The Young's modulus was determined as slope in the linear regime at a strain of 6-8%. The initial part of the stress-strain curve (strain=0-5%) shows tailing due to the slightly uneven surface of the sample and was thus not considered.

The gel structure is highly reinforced by the protein scaffold. The gel could be further loaded with an active substance for pharmaceutical or agrochemical applications. The protein sponge provides mechanical stability and integrity. The elasticity of the sponge structure when being filled with an aqueous liquid allows for multiple loading and release cycles.

### Example 6

Production of Sponge Particles without Mould

40 wt % whey protein isolate was dispersed in tap water and hydrated overnight. Foaming with a kitchen whipping machine (Kitchen Aid) resulted in a gas volume fraction of approximately 65 vol %. Drops of 5-10 mm diameter of the foam were deposited onto a Telfon plate and dried at 150 W and 60° C. for 30 minutes with an additional beaker inside the oven cavity filled with 500 mL water for higher humidity.

The resulting stiff sponge structure particles absorbed water without disintegrating and softened when filled with water. When in contact with oil, the sponge structures absorbed the oil without disintegrating but remained brittle.

### Example 7

Production of Sponge Spheres in Moulds

The foam was prepared as described in Example 6. The foam was transferred into praline moulds with a diameter of about 15-30 mm and dried at 150 W and 60° C. for 30 minutes with an additional beaker inside the oven cavity filled with 500 mL water for higher humidity. The resulting stiff sponge structure spheres absorbed water without disintegrating and softened when filled with water. When in contact with oil, the sponge structures absorbed the oil without disintegrating but remained brittle.

### Example 8

Production of a Partly Dried Sponge-Like Formulation

The foam was prepared as described in Example 6. The foam was transferred into cylinders as in Example 1 and dried at 100 W and 60° C. for 15 minutes. The resulting sponge formulation had a moisture content of approximately 50% and absorbed water and oil without disintegrating. The liquid absorption capacity for water was approximately 6 g/g sample and for oil 3 g/g sample.

List of literature
(1) CN 108273476 A
(2) WO 2015/056273
(3) Duan, Bo, et al. “Hydrophobic modification on surface of chitin sponges for highly effective separation of oil” ACS applied materials & interfaces 6.22 (2014): 19933-19942
(4) Jiang, Feng, and You-Lo Hsieh. “Amphiphilic superabsorbent cellulose nanofibril aerogels”, Journal of Materials Chemistry A 2.18 (2014): 6337-6342
(5) Zhu, Haiguang, et al. “A robust absorbent material based on light-responsive superhydrophobic melamine sponge for oil recovery.” Advanced Materials Interfaces 3.5 (2016): 1500683
(6) M. Betz, C. A. Garcia-Gonzalez, R. P. Subrahmanyam, I. Smirnova, U. Kulozik, Preparation of novel whey protein-based aerogels as drug carriers for life science applications, The Journal of Supercritical Fluids, Volume 72, 2012, Pages 111-119, ISSN 0896-8446
(7) Selmer, Ilka, et al. “Development of egg white protein aerogels as new matrix material for microencapsulation in food.” The Journal of Supercritical Fluids 106 (2015): 42-49
(8) V. Perez-Puyana, M. Felix, A. Romero, A. Guerrero, Development of eco-friendly biodegradable superabsorbent materials obtained by injection moulding, Journal of Cleaner Production, Volume 198, 2018, Pages 312-319, ISSN 0959-6526

1. A method of making a porous sponge-like formulation that can absorb water and oil, said method comprising the steps:
a. Preparing a 5-60 wt % protein dispersion in aqueous liquid, preferably in water;
b. Dispersing gas in the protein dispersion to form a foam structure;
c. Optionally moulding or shaping to form a foam structure;
d. Expansion of the foam structure;
e. Volumetric heating induced water evaporation and protein denaturation;
f. Optionally drying; and
g. Optionally cutting into pieces.

2. The method according to claim 1, wherein about 10-50 wt % protein dispersion is prepared in water.

3. The method according to claim 1, wherein gas is dispersed in the protein dispersion using a rotating membrane foaming device.

4. The method according to claim 1, wherein said foam structure has a gas volume fraction of 10-90 vol %, preferably 40-80 vol %, most preferably 60-75 vol %.

5. The method according to claim 1, wherein heating and/or drying is volumetric preferably through application of electromagnetic waves, preferably microwave, most preferably with superposition of convection heating.

6. The method according to claim 1, wherein the temperature gradient between the core and the surface layer of the foam structure is between −0.1 to 0.3, preferably from −0.1 to 0.1 and the foam structure temperature is above the denaturation temperature of the protein during heating.

7. The method according to claim 1, wherein a vacuum is applied before and/or during drying which is between 10-800 mbar, preferably 50-500 mbar, more preferably between 100-300 mbar.

8. The method according to claim 1, wherein said porous sponge-like formulation has open pores having an average pore size diameter of up to 500 microns, preferably up to 200 microns.

9. A porous sponge-like formulation comprising protein, obtained by a method according to claim 1.

10. A porous sponge-like formulation that can absorb water, oil and organic solvent and comprises protein, wherein the said formulation has a porosity of between 10-95 vol %, and wherein said formulation can absorb water and oil without disintegrating or dissolving to an extent of less than 10 wt %.

11. The porous sponge-like formulation according to claim 9, wherein said formulation further comprises soluble and/or non-soluble fibre and/or polysaccharides.

12. The porous sponge-like formulation according to claim 9, wherein said formulation is capable of absorbing water and oil at substantially the same velocity, preferably up to 5 mm/s, and wherein the absorbed water, oil and organic solvent can be removed by compression or suction and re-absorbed into the same said formulation.

13. The porous sponge-like formulation according to claim 9 wherein said formulation is capable of absorbing water with a temperature of 0-100° C. to an extent that up to 140%, preferably 160%, of pore volume is filled with water due to additional structural swelling effects.

14. The porous sponge-like formulation according to claim 9 wherein said formulation is capable of absorbing oil with a temperature of 0-200° C. to an extent that 90% of pore volume, preferably up to 95% of pore volume, most preferably up to 100% of the pore volume is filled with oil.

15. The porous sponge-like formulation according to claim 9 wherein said formulation is elastically and plastically deformable after absorption of water and is brittle in substantially dry state and after absorption of oil.

16. Use of the porous sponge-like formulation according to claim 9 in a product, applied in one of the areas of medicine, pharmacology, biotechnology, chemistry as well as in home care, wound care, (agro-) environmental and construction material applications.

17. Use of the porous sponge-like formulation according to claim 9 as filter and/or absorber for the cleaning of watery and/or oil-based fluid systems and/or water/oil mixtures.

18. Use of the porous sponge-like formulation according to claim 9 as immobilization carrier for microorganisms in biotechnological, pharmaceutical and/or medical applications

19. Use of the porous sponge-like formulation according to claim 9 for wound care treatment with high absorbance for wound secretion fluid and tailored release of substances or drugs for the wound related treatment.

20. Use of the product according claim 9 for medical surgery applications to enable larger amounts of blood or secretion fluid uptake.

21. Use of the product according to claim 9 for cosmetics applications with water/oil-based liquid and skin care ingredients or skin cleaning fluids release from and/or uptake into the sponge-like product.

22. Use of the product according to claim 9 for the encapsulation of active/functional ingredients from the categories: flavors, aromas, micronutrients, antioxidants, agro-chemicals, chemicals, washing agents, drugs, pre-/probiotic cultures, skin care components, cleaning components.

23. Use of the product according to claim 9 as template (3) for cell culturing

24. A product comprising the foamed sponge-like formulation of claim 9.
